(19) United States
(12) Patent Application Publication
(10) Pub. No.: US 2008/0112886 A1
(43) Pub. Date: May 15, 2008

(54) ENGINEERING SHAPE OF POLYMERIC MICRO- AND NANOPARTICLES

(75) Inventors: Samir Mitragotri, Goleta, CA (US); Julie A. Champion, Santa Barbara, CA (US); Yogesh K. Katare, Goleta, CA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: The Regents of the University of California

(21) Appl. No.: 11/851,974

(22) Filed: Sep. 7, 2007

Related U.S. Application Data
(60) Provisional application No. 60/825,085, filed on Sep. 8, 2006.

(51) Int. Cl. A61K 9/51; C08F 6/00; A61K 49/00; A61K 9/50

(52) U.S. Cl. 424/9.1; 528/502 R; 424/501

(57) ABSTRACT

Compositions containing polymeric micro- and nanoparticles with non-spherical shapes and methods for making and using such particles are described herein. The particles have a size range from an average diameter of about Compositions containing polymeric micro- and nanoparticles with non-spherical shapes and methods for making and using such particles are described herein. The particles have one or more dimensions ranging from about 5 nm to about 100 μm, preferably about 100 nm to 10 μm. The particles can have any of a wide variety of non-spherical shapes. The particles are generally formed by manipulation of spherical particles embedded in a polymeric film. A wide variety of resulting shapes can be made. The resulting shape is a function of whether the films are manipulated in a first and/or second dimension, and the processes used to liquefy the microparticles. Variations of the method of manufacture may be used to generate particles having the desired shapes in large, reproducible quantities. The resulting non-spherical shaped particles can be used to alter uptake by phagocytic cells and thereby clearance by the reticuloendothelial system.

### CROSS-REFERENCE TO RELATED APPLICATIONS

This application is a non-provisional application of U.S. Ser. No. 60/825,085, filed Sep. 8, 2006. The disclosure in the application listed above is herein incorporated by reference.

### GOVERNMENT SUPPORT

The United States Government has certain rights in this invention by virtue of National Institutes of Health grant No. 1VO1 HL080718 to Samir Mitragotri.

### FIELD OF THE INVENTION

The present invention relates to polymeric micro- and nanoparticles with non-spherical shapes.

### BACKGROUND OF THE INVENTION

Polymeric micro and nanoparticles have found numerous applications in diverse fields such as drug delivery (Stolnick, et al, Adv. Drug Delivery Rev., 16:195-214 (1995)), advanced materials (Subramanian, et al., Adv. Mater., 11:1261-1265 (1999)), personal care (Luppi, et al., J. Pharm. Pharmacol., 56:407-411 (2004)) and medical imaging (Chen, et al., Magn. Reson. Med., 53:614-620 (2005)). Significant attention has been paid to engineering particle properties such as size, surface chemistry, and to a much lesser extent, shape, to optimize particle function. The relatively few studies on particle shape are largely due to difficulties in synthesizing precisely shaped polymeric particles.

A major difficulty with the use of micro- and nanoparticles for in vivo applications such as drug delivery, immunization and diagnostics, is phagocytosis by professional phagocytes and components of the reticuloendothelial system. Phagocytosis limits the circulation half-life of micro- and nanoparticles and impedes tissue-specific targeting. While many studies have investigated the effects of size and surface chemistry on phagocytosis of micro- and nanoparticles, the effect of particle shape on phagocytosis is not known.

Previous approaches to making non-spherical polymeric particles have made use of self-assembly of spherical nanoparticles (Manoharan, et al., Science, 301:483-487 (2003); Yin, et al, Adv. Mater., 13:267-271 (2001); Velev, et al., Science, 287:2240-2243 (2000)), photolithography (Dendukuri, et al., Nature Mater., 5:365-369 (2006)), microfluidics (Xu, et al., Angew. Chem. Int. Ed., 44:724-728 (2005); Dendukuri, et al., Langmuir, 21:2113-2116 (2005)), photopolymerization (Fernandez-Nieves, et al., Adv. Mater., 17:680-684 (2005); Brown, et al., Phys. Rev., 62:951-960 (2000)), and stretching of spherical particles (Ho, et al, Colloid Polym. Sci., 271:469-479 (1993); Lu, et al., Adv. Mater., 13:271-274 (2001)). Collectively, these methods have produced particles of several distinct shapes. Some of these methods provide advantages such as scalability, high throughput, and precise control over particle shape. However, these methods also suffer from drawbacks including cost, limitations on particle size, low throughput, and limited ability to sculpt particles in three dimensions.

It would be advantageous to provide polymeric micro- or nanoparticles which are non-spherical in shape.

It would also be advantageous to provide a method for making such particles which provides for scalability, high throughput, versatility to control the shape of non-spherical particles, and/or precise control over the shape of such non-spherical particles.

Therefore, it is an object of the invention to provide polymeric particles in the micrometer and nanometer size ranges which are non-spherical in shape.

It is a further object of the invention to provide an improved method for producing polymeric particles in the micron and submicron size that enables manipulation of the particles into non-spherical shapes.

It is a further object of the invention to provide polymeric particles in the micrometer and nanometer size ranges which have non-spherical shapes effective to decrease phagocytosis.

It is an even further object of the invention to provide polymeric particles in the micrometer and nanometer size ranges which are non-spherical in shape that can be used for a variety of applications, including drug delivery, therapy, diagnosis, prophylaxis, and immunization.

### SUMMARY OF THE INVENTION

Compositions containing polymeric micro- and nanoparticles with non-spherical shapes and methods for making and using such particles are described herein. The particles have an one or more dimensions ranging from about 5 nm to about 100 μm, preferably about 100 nm to 10 μm. The particles can have any of a wide variety of non-spherical shapes. The particles are generally formed by manipulation of spherical particles embedded in a polymeric film. A wide variety of resulting shapes can be made. The resulting shape is a function of whether the films are manipulated in a first and/or second dimension, and the processes used to liquefy the microparticles. Variations of the method of manufacture may be used to generate particles having the desired shapes in large, reproducible quantities. The resulting non-spherical shaped particles can be used to alter uptake by phagocytic cells and thereby clearance by the reticuloendothelial system.

### BRIEF DESCRIPTION OF THE DRAWINGS

FIG. 1 depicts schemes A, B and C which may be used to shape micro- and nanoparticles embedded in a polymeric film.

FIGS. 2a-z depicts the shapes of the particles formed in the examples: (a) spheres, (b) rectangular disks, (c) high aspect ratio rectangular disks, (d) rods, (e) high aspect ratio rods, (f) worms, (g) oblate ellipses (h) prolate ellipses, (i) elliptical disks, (j) UFOs, (k) circular disks, (l) barrels, (m) bullets, (n) pills, (o) pulleys, (p) bi-convex lenses, (q) ribbons, (r) ravioli, (s) flat pills, (t) bicones, (u) diamond disks, (v) emarginate disks, (w) elongated hexagonal disks, (x) tacos, (y) wrinkled prolate ellipsoids, (z) wrinkled oblate ellipsoids and (aa) porous elliptical disks.

FIG. 3a is a schematic diagram illustrating how a macrophage membrane travels tangentially around an elliptical disk. FIG. 3b is a graph of membrane velocity as a function of Ω, a dimensionless parameter that depends on the shape of the particle at its point of attachment to the membrane of the macrophage.

FIG. 4 is a phase diagram of phagocytosis with Ω and dimensionless particle volume V* (particle volume divided by 7.5 μm radius spherical cell volume) as governing parameters (n=5 for each point).

### DETAILED DESCRIPTION OF THE INVENTION

### I. Compositions

The compositions contain non-spherical micro- or nanoparticles. The non-spherical particles are prepared by embedding spherical micro- or nanoparticles in a polymer film and manipulating, such as by stretching, the film to alter the shape of the particles. In order to alter the shape of the particles, the particles have to adhere to the film so that when the film is stretched the particle is stretched as well. Adherence may be by hydrogen bond formation, or other non-covalent interactions (e.g. ionic bonds, van der Waals interactions, etc).

A. Size of Micro- and Nanoparticles

The non-spherical particles have one or more dimensions ranging from about 5 nm to 100 microns, preferably from about 5 nm to 10 microns, more preferably from about 10 nm to 5 microns, and most preferably from about 30 nm to 2 microns. In one embodiment, the particles have one or more dimensions in the submicron range, i.e. less than 1 micron, such as from 200 nm to 800 nm.

B. Shape of Micro- and Nanoparticles

Particles may be in the form of any non-spherical shape. As generally used herein, “non-spherical” is used to describe particles having at least one dimension differing from another dimension by a ratio of at least 1:1.10. In one embodiment, the non-spherical particles have at least one dimension which differs from another dimension by a ratio of at least 1:1.25. A wide variety of shapes are considered “non-spherical” shapes. For example, as shown in FIGS. 2a-z and 2aa, non-spherical particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those illustrated in the figures are also within the scope of the definition for “non-spherical” shapes.

C. Polymers in Nano- or Microparticles

Any synthetic or natural polymer can be used to form the micro- and nanoparticles. The polymer, copolymer, or blend of polymers used to form the nano- or microparticles is referred to herein as the “particle polymer”. In one embodiment, the particle polymer is chosen for a particular property, such as biocompatibility, biodegradability, bioadhesivity, etc. The microparticle should be capable of adhering to the polymer film via non-covalent interactions including, but not limited to, hydrogen bonding.

Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and derivatives, copolymers and blends thereof derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), and derivatives, copolymers and blends thereof. Examples of non-biodegradable polymers include ethylene vinyl acetate, poly(meth)acrylic acid, polyamides, and derivatives, copolymers and mixtures thereof. Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), and derivatives, blends and copolymers thereof.

Examples of natural polymers include proteins such as albumin, collagen, gelatin and prolamines like zein, and polysaccharides such as alginate, cellulose derivatives and polyhydroxyalkanoates like polyhydroxybutyrate and polyhydroxybutyrate-valerate and blends thereof.

Bioadhesive polymers include polyanhydrides, and polymers and copolymers of acrylic acid, methacrylic acid, and their lower alkyl esters, for example polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

As used herein, “derivatives” include polymers having substitutions, additions of chemical groups and other modifications routinely made by those skilled in the art.

The in vivo stability of the matrix can be adjusted during the production by using a copolymer, such as one which contains polyethylene glycol (PEG), e.g. polymers such as polylactide-co-glycolide copolymerized with PEG. PEG, if exposed on the external surface of the micro- or nanoparticles, may elongate the time these materials circulate in vivo since it is hydrophilic.

D. Excipients and Additives

A variety of excipients and additives may optionally be present in the micro- or nano-particles. These include adjuvants such as, bacterial toxins or membrane permeabilizing agents, such as surfactants, fatty acids and fatty esters.

E. Targeting Ligands

The particles may further contain a targeting moiety to facilitate targeting of the micro- or nano-particles to a specific site in vivo. The targeting moiety may be any moiety that is conventionally used to target an agent to a given in vivo site such as an antibody, a receptor, a ligand, a peptidomimetic agent, an aptamer, a polysaccharide, a drug or a product of phage display.

F. Labels

The micro- or nano-particles may be conjugated to a detectable label, for example, a radiolabel, chemiluminescent or fluorescent label, or immunolabel.

### II. Methods for Making Non-Spherical Micro- and Nanoparticles.

The non-spherical microparticles are typically formed by manipulation of spherical micro- or nanoparticles. The micro- or nano-particles can be applied to a polymeric film as a liquid (i.e. droplets), prior to stretching the film. Alternatively, the particles can be added as a solid to the polymeric film. In this embodiment, the polymeric film can be stretched creating voids around the, micro- or nano-particles, and then the micro- or nano-particles can be liquefied.

A. Polymeric Film

The polymeric film can be in the form of a film or a block. The film must be in the form of a solid in order to allow for it to be manipulated, such as by stretching. The particles can be in a liquid form initially, e.g. in the form of droplets, or in the form of a solid, e.g. particles, which are subsequently liquefied following application to the polymeric film.

As used herein, “film” does not refers to both thin films, with thicknesses ranging from about 10 microns to 500 microns, and blocks of polymer, with thicknesses ranging from 500 microns to about 10 cm, which can be stretched using the same methods, for example, a 10 cm×10 cm×20 cm block.

Any synthetic or natural polymer can be used to form the polymeric film. The polymer, copolymer, or blend of polymers used to form the nano- or microparticle the polymeric film is referred to herein as the “film forming polymer”. Two important criteria for selecting the film forming polymer in which the particles will be embedded is immiscibility and stretchability. In order to form the non-spherical particles, the particle polymer must be immiscible in the film forming polymer and the particle polymer and the film forming polymer should not be soluble in the same solvents. The polymeric film should be sufficiently stretchable such that the nano- and microparticles can be manipulated to form non-spherical shapes. Stretchability can be modified by incorporation of additives into the polymer, such as plasticizers.

Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and derivatives, copolymers and blends thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), and derivatives, copolymers and blends thereof. Examples of non-biodegradable polymers include ethylene vinyl acetate, poly(meth)acrylic acid, polyamides, and derivatives, copolymers and mixtures thereof. Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), and derivatives, blends and copolymers thereof. In the preferred embodiment, the film forming polymer is polyvinyl alcohol.

Examples of natural polymers include proteins such as albumin, collagen, gelatin and prolamines like zein, and polysaccharides such as alginate, cellulose derivatives and polyhydroxyalkanoates like polyhydroxybutyrate and polyhydroxybutyrate-valerate and blends thereof.

As used herein, “derivatives” include polymers having substitutions, additions of chemical groups and other modifications routinely made by those skilled in the art.

i. Plasticizers

One or more plasticizers may be added to the film forming polymer film to facilitate stretching. Representative classes of plasticizers include: abietates, adipates, alkyl sulfonates, azelates, benzoates, chlorinated paraffins, citrates, energetic plasticizers, epoxides, glycol ethers and their esters, glutarates, hydrocarbon oils, isobutyrates, oleates, pentaerythritol derivatives, phosphates, phthalates, polymeric plasticizers, esters, polybutenes, ricinoleates, sebacates, sulfonamides, tri- and pyromellitates, biphenyl derivatives, calcium stearate, carbon dioxide, difuran diesters, fluorine-containing plasticizers, hydroxybenzoic acid esters, isocyanate adducts, multi-ring aromatic compounds, natural product derivatives, nitrites, siloxane-based plasticizers, tar-based products and thioesters. An exemplary plasticizer is glycerol at a concentration of about 2% w/v.

B. Methods for Making Spherical Nano- and Microparticles

There are several well-known processes whereby spherical nano- or microparticles can be made, including, for example, spray drying, interfacial polymerization, hot melt encapsulation, phase separation encapsulation, spontaneous emulsion, solvent evaporation microencapsulation, solvent removal microencapsulation, coacervation, low temperature microsphere formation, and phase inversion nanoencapsulation (“PIN”).

i. Spray Drying

In spray drying, the core material to be encapsulated in the resulting micro- or nanoparticles is dispersed or dissolved in a solution. Typically, the solution is aqueous and preferably the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

ii. Interfacial Polycondensation

Interfacial polycondensation is used to microencapsulate a core material in the following manner. One particle monomer and the core material are dissolved in a solvent. A second particle monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

iii. Hot Melt Encapsulation

In hot melt microencapsulation, the core material (to be encapsulated) is added to molten particle polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

iv. Solvent Evaporation Microencapsulation

In solvent evaporation microencapsulation, the particle polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in the particle polymer. The particle polymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the particle polymer to phase separate and migrate towards the surface of the core material droplets. This phase-separated solution is then transferred into an agitated volume of nonsolvent, causing any remaining dissolved particle polymer to precipitate and extracting any residual solvent from the formed membrane. The result is a microcapsule composed of a particle polymer shell with a core of liquid material.

v. Solvent Removal Microencapsulation

In solvent removal microencapsulation, the particle polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the particle polymer and the particle polymer/solvent solution is immiscible in the oil. The organic solvent is removed by diffusion into the oil phase while continuing to stir. Solvent removal results in precipitation of the particle polymer, forming solid microcapsules containing core material.

vi. Phase Separation Microencapsulation

In phase separation microencapsulation, the material to be encapsulated is dispersed in a particle polymer solution with stirring. While continually stirring to uniformly suspend the material, a nonsolvent for the polymer is slowly added to the solution to decrease the polymer's solubility. Depending on the solubility of the particle polymer in the solvent and nonsolvent, the particle polymer either precipitates or phase separates into a polymer rich and a polymer poor phase. Under proper conditions, the particle polymer in the polymer rich phase will migrate to the interface with the continuous phase, encapsulating the core material in a droplet with an outer polymer shell.

vii. Spontaneous Emulsification

Spontaneous emulsification involves solidifying emulsified liquid particle polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

viii. Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation, compositions comprised of two or more phases and known as coacervates may be produced. The ingredients that comprise the two phase coacervate system are present in both phases; however, the colloid rich phase has a greater concentration of the components than the colloid poor phase.

ix. Phase Inversion Nanoencapsulation (“PIN”)

PIN is a nanoencapsulation technique which takes advantage of the immiscibility of dilute polymer solutions in select “non-solvents” in which the polymer solvent has good miscibility. The result is spontaneous formation of nanospheres (less than 1 μm) and microspheres (1-10 μm) within a narrow size range, depending on the concentration of the initial polymer solution, the molecular weight of the polymer, selection of the appropriate solvent-non-solvent pair and the ratio of solvent to non-solvent. Encapsulation efficiencies are typically 75-90% and recoveries are 70-90% and bioactivity is generally well-maintained for sensitive bioagents.

“Phase inversion” of polymer solutions under certain conditions can bring about the spontaneous formation of discreet microparticles. PIN is essentially a one-step process, is nearly instantaneous, and does not require emulsification of the solvent. Under proper conditions, low viscosity polymer solutions can be forced to phase invert into fragmented spherical polymer particles when added to appropriate nonsolvents.

Phase inversion phenomenon has been applied to produce macro- and micro-porous polymer membranes and hollow fibers, the formation of which depends upon the mechanism of microphase separation. A prevalent theory of microphase separation is based upon the belief that “primary” particles form of about 50 nm diameter, as the initial precipitation event resulting from solvent removal. As the process continues, primary particles are believed to collide and coalesce forming “secondary” particles with dimensions of approximately 200 nm, which eventually join with other particles to form the polymer matrix. An alternative theory, “nucleation and growth”, is based upon the notion that a polymer precipitates around a core micellar structure (in contrast to coalescence of primary particles).

The process results in a very uniform size distribution of small particles forming at lower polymer concentrations without coalescing. By adjusting polymer concentration, polymer molecular weight, viscosity, miscibility and solvent:nonsolvent volume ratios, the interfibrillar interconnections characteristic of membranes using phase inversion are avoided, with the result being that microparticles are spontaneously formed. These parameters are interrelated and the adjustment of one will influence the absolute value permitted for another.

In the preferred processing method, a mixture is formed of the agent to be encapsulated, a particle polymer and a solvent for the polymer. The agent to be encapsulated may be in liquid or solid form. It may be dissolved in the solvent or dispersed in the solvent. The agent thus may be contained in microdroplets dispersed in the solvent or may be dispersed as solid microparticles in the solvent. The phase inversion process thus can be used to encapsulate a wide variety of agents by including them in either micronized solid form or else emulsified liquid form in the particle polymer solution

x. Melt-Solvent Evaporation Method

In the melt-solvent evaporation method, the particle polymer is heated to a point of sufficient fluidity to allow ease of manipulation (for example, stirring with a spatula). The temperature required to do this is dependent on the intrinsic properties of the particle polymer. For example, for crystalline polymers, the temperature will be above the melting point of the polymer. After reaching the desired temperature, the agent to be encapsulated is added to the molten polymer and physically mixed while maintaining the temperature. The molten particle polymer and agent to be encapsulated are mixed until the mixture reaches the maximum level of homogeneity for that particular system. The mixture is allowed to cool to room temperature and harden. This may result in melting of the agent in the polymer and/or dispersion of the agent in the polymer. The process is easy to scale up since it occurs prior to encapsulation. High shear turbines may be used to stir the dispersion, complemented by gradual addition of the agent into the polymer solution until the loading is achieved. Alternatively the density of the polymer solution may be adjusted to prevent agent from settling during stirring.

C. Methods for Altering Shape of Nano and Microparticles

The non-spherical particles are formed by manipulation of spherical particles. The method of making non-spherical shapes involves embedding the particles in a polymeric film and manipulating the film using various permutations of the following steps: (1) liquefaction of the particles or the film, (2) stretching or other physical manipulation of the polymeric film, and (3) solidification of the particles or film.

The order of the steps is important as switching the order of the steps can result in the formation of different shapes. Thus, steps 1-3 can be re-ordered to expand the diversity of the non-spherical shapes that are formed. For example, when steps 1 and 2 are re-ordered, stretching of the film generates air-filled voids around the solid particle, which can then be filled by liquefaction of the embedded particle. The manner in which the particle fills the void upon liquefaction can be a function of the temperature (“heat-induced liquefaction”) or solvent (“solvent-induced liquefaction”) used to liquefy the particle.

Shapes produced by any order of these steps can, in turn, be used as new starting materials for another round of manipulation using any order of these steps, further expanding the shape diversity. Thus, various combinations of these steps performed in tandem can produce a large number of different shapes from the same starting material.

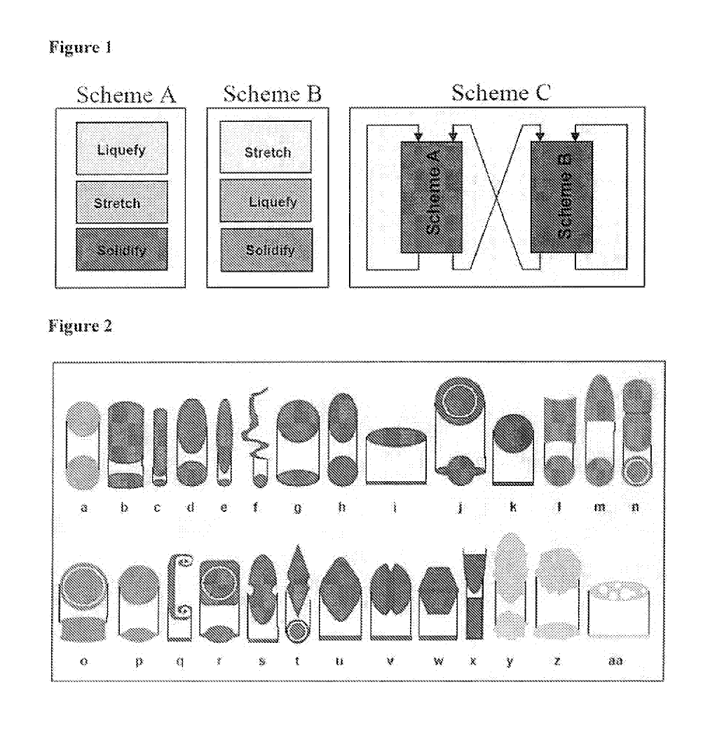

Various schemes can be used for making different shapes. Exemplary schemes are depicted in FIG. 1. Scheme A involves the following order of steps, (1) liquefaction of the particles, (2) stretching of the film, and (3) solidification of the particles. Scheme B involves the following order of steps: (1) stretching of the film, (2) liquefaction of the particles, and (3) solidification of the particles. Scheme C involves all possible sequential combinations of Scheme A and Scheme B, wherein the particles are repeatedly stretched to generate complex shapes. Thus, the shapes produced in Scheme A and/or B, in turn, can be used as new starting materials to further expand the shape diversity, as shown in Scheme C.

Optionally, the film may be reinforced after a round of liquefaction and stretching by immersing the film in a solution of the film forming polymer.

One advantage of this method is that it uses routine, inexpensive laboratory chemicals and equipment. Another advantage of this method is that it can reproducibly produce at least 20 distinct shapes. A further advantage of this method is that can be applied to particles with dimensions on the micro- and nano-scales. A further advantage of this method is that it results in high throughput. Scale-up production with larger and more stretching devices is practical.

i. Liquefaction of the Particles or the Film

In a first embodiment, particles first are embedded into a polymeric film. In this step, a film forming polymer is dissolved in an appropriate solvent (e.g. water in the case of polyvinyl alcohol) at a concentration effective to produce a desired film thickness. Optionally, a plasticizer may be added to the solution. Spherical micro- or nanoparticles are then added to the solution in solid or liquid form. In a preferred embodiment, polymeric micro- or nanoparticles are added to a film forming polymer solution at a preferred concentration of 0.001-0.004% (wt particles/wt film polymer). Particle concentration can be increased so long as the particles are not touching each other in the film, which can result in large conglomerates upon liquefaction.

Liquefaction of the embedded particles may be induced using heat or an appropriate solvent. The resulting particle shape is affected by the method of liquefaction. For example, in the case of polystyrene, replacing heat with toluene as the mode of liquefaction resulted in particles with entirely different shapes than when the particles were liquefied by heating. It is believed that the difference in viscosity of the polystyrene when dissolved in toluene versus when it is heated results in the difference in shape.

The solution of polymer and particles is then poured onto a fiat surface and dried to a desired thickness to form a film. The thickness of the resulting film can range from about 10 microns to several centimeters or more. In general, thinner films result in the formation of flatter particles. It is believed that the width of the polymer film does not impact the final shape of the manipulated nano- and microparticles.

ii. Stretching of the Film

The polymeric film transfers strain to the particles during stretching, and also acts as a support to trap the liquefied particles. Adhesion between the particles and film causes particles to deform in response to film stretching. Since many polymers have glass transition temperatures (Tg) above room temperature, many polymeric films stretch very little at room temperature. Treatments may be done to the polymeric film to facilitate stretching. For example, the film can be heated, or a plasticizer can be added to the film.

The film may be stretched in one dimension or in two dimensions. Stretching in one dimension may be achieved, for example, by attaching the film to two opposing blocks which are mounted in a screw, which when turned, separates the blocks (a “1-D stretcher”). Stretching in two dimensions may be achieved, for example, using two sets of such opposing blocks which move simultaneously (a “2-D stretcher”). As described in the examples, the polymeric film is typically cut into sections and mounted on a 1-D or 2-D stretcher. The extent of stretching the polymeric film may be varied from as little as 1.1-fold to as much as 13-fold, or even greater, depending on the objective.

In one embodiment, stretching in one dimension or two dimensions may be performed with the film exposed to air. In another embodiment, the film may be stretched while immersed in hot oil or another heated immiscible solvent (e.g. toluene, methylene chloride, chloroform, or any other organic solvent in which the particle polymer is soluble) effective to liquefy the embedded particles. In another embodiment, the film may be stretched while immersed in a solvent effective to liquefy the embedded particles. The solvent may be removed by drying the film and extracting residual solvent. Alternatively, the air-drying step may be skipped and solvent may be extracted directly.

In case of heat-stretching, the film is immersed in hot oil for a suitable time period to heat the polymeric film and liquefy the particles, such as 5 minutes, and the film is stretched while still in the oil. The temperature of the oil is controlled between 120° C. and 155° C., depending on the desired shape.

In schemes in which the films are stretched prior to liquefaction of the particles, the films may be heated after stretching for a suitable time to liquefy the particles. In both cases, following liquefaction of the particles, and optionally stretching, the film is removed from the oil and allowed to cool in air for a suitable period of time to harden the particles.

In case of stretching a polymeric film in an immiscible solvent, such as toluene, the film is immersed in solvent for a suitable period of time to liquefy the embedded particles, e.g. 3 hours, and the film is stretched while still in solvent. In schemes in which the films are stretched prior to liquefaction of the particles, the films may also soaked be soaked in an immiscible solvent for a suitable period of time to liquefy the particles. After immersion in the immiscible solvent, the film is typically removed from the solvent and air dried for a suitable period of time to evaporate off most of the solvent. Then the film is soaked in isopropanol or another suitable solvent for a suitable period of time to extract residual amounts of the immiscible solvent.

In another embodiment, to induce formation of holes in particles, the step of air drying is skipped, and the polymeric films are placed directly in a suitable solvent to extract the immiscible solvent following the stretching step.

In several embodiments, such as described in the Examples, the film may be stretched sequentially in multiple dimensions or multiple times in the same dimension.

Final particle shape is dictated by the material properties of the film (T_{g} and thickness), material properties of the particles (T_{g} and viscosity), interactions between particles and film (adhesion strength), and the operating parameters (extent and dimensionality of stretching). The range and combinations of these characteristics give rise to a diverse group of particle shapes. Particle volume remains constant during stretching, governed entirely by the volume of the initial micro- or nanosphere. Thus, size and shape of particles can be independently controlled.

In some embodiments, the film is reinforced after one round of liquefaction and stretching. For example, the films can be reinforced by sandwiching the film between two layers of a solution of the same film polymer and allowing the film to dry in air for a suitable period of time, such as 24 hours.

iii. Solidification of the Particles or Film

Solidification of the particles generally occurs by cooling or solvent extraction and recovery of particles by dissolution of the film. Re-solidifying the particles after manipulation, by solvent extraction or cooling, sets their new shape.

In one embodiment, following completion of particle shaping step(s), the polymeric films are dissolved in a miscible solvent. The shaped micro- or nanoparticles may be washed in the same solvent multiple times to remove excess film polymer.

Particle shapes may be observed and characterized using conventional methods.

The isolated particles can be chemically modified after formation or by incorporation during particle formation. The particles may be coated with a material, such as palladium (e.g. Hummer® 6.2 Sputtering System, Anatech Ltd., Union City, Calif.), and imaged using scanning electron microscopy ((e.g. Sirion® 400 Scanning Electron Microscope (FEI Company, Hillsboro, Oreg.)). Particle dimensions can be measured can be measured by various methods, including light scattering, electron micrography etc. For example, particle dimensions can be determined from micrographs, such as by using Metamorph® image acquisition and analysis software (Universal Imaging Systems, Downingtown, Pa.).

### III. Uses for Non-Spherical Micro- and Nanoparticles

The non-spherical micro- and/or nanoparticles may be used in many applications including therapeutic applications, such as drug delivery, diagnostic applications and immunization.

For example, the non-spherical micro- and/or nanoparticles produced according to the methods described herein may be used in the delivery of drugs and vaccines. Any suitable delivery means may be used, including but not limited to oral, inhalation, nasal, subcutaneous and other routes. The shapes may be selected to alter uptake by phagocytic cells and thereby clearance by the reticuloendothelial system. For example, the shape may be selected to control uptake by macrophages, such as by reducing or decreasing the rate of phagocytosis.

As shown in the Examples, the overall process of phagocytosis is a result of the complex interplay between shape and size. In particular, the Examples show that the distinction between phagocytosis and spreading is defined by the shape of the particle that interacts with the cell, Ω (see FIG. 4). Macrophages phagocytosed particles as large as themselves when the portion of the particle approached the cell from the preferred orientation, i.e. Ω≦45°. However, when the particle approached from the undesired orientation, i.e. Ω>45°, the cells did not internalize the particles, even when the particles were quite small, such as with volumes as small as 0.2% of the cell volume.

Non-spherical particles also have applications as standards for shape analysis. Numerous pharmaceuticals, biotechnology products, abrasives, ceramics, explosives and toners utilize nano- and micro-size powders. Significant efforts are currently spent in characterizing size and shape of nano- and micro-size powders through methods such as laser diffraction and image analysis. Well-defined particles of various shapes play a critical role as calibration standards in such methods. Currently, there is a scarcity of such standards and the particles described in this study can be used to establish or improve calibration standards for nano- and micro-size particles.

Particles with different shapes formed by the methods described herein also have numerous applications in fundamental studies in biomedicine. For example, artificial joint prosthesis leads to polymeric debris in joints, which activates release of inflammatory mediators and promote osteolysis. The debris contains particles of various shapes and texture and research suggests that particle shape and surface texture may be key determinants of inflammation. However, this problem can only be understood by using cellular response to uniform and well-controlled particles of diverse shapes. A better understanding of material-cell interaction could lead to fabrication of novel biomaterials which can direct cells morphology and proliferation, and eventually release biological signals to properly conduct tissue formation.

In another application, migration of bacteria in groundwater is a major environmental challenge. Most bacteria are non-spherical and hence spherical particles cannot be used for these studies. Non-spherical shaped micro- and nanoparticles produced according to the methods described herein are useful in studies for migration of bacteria and ground water. Non-spherical shaped micro- and nanoparticles produced according to the methods described herein are useful for studies of environmental debris (dust, pollen, asbestos etc.) whose migration into lungs and eventual toxicity depends on their aerodynamic properties, which in turn are related to their size, shape and surface texture.

Non-spherical shaped micro- and nanoparticles produced according to the methods described herein also provide model shapes for human cells and organelles (for example, oblate ellipsoidal platelets, discoidal erythrocytes, and prolate ellipsoidal mitochondria) whose transport properties in blood or within cells are of significant fundamental interest.

Non-spherical micro- and nanoparticles produced according to the methods described herein are also useful models to study important physical problems, for example, self-assembly of nematic crystals. Since the method disclosed herein produces highly uniform particles, many of them, especially rod-shaped particles, readily organize into structure that resemble nematic crystals. More importantly, these particles can be observed using optical microscopy which facilitate their studies.

Non-spherical micro- and nanoparticles produced according to the methods described herein also have unique opportunities for studying challenging problems in fluid dynamics. Flow behavior of non-spherical particles has extensive implications in fundamental understanding and technological applications. These particles make ideal fluids and hence provide ideal probes for understanding the role of shape in rheology. These particles can also be used to study additional fundamental, shape-sensitive phenomena in physics, for example, light scattering, interfacial adsorption, packing densities, sedimentation, and fluidization and granular flows. Many of these phenomena, for example, light scattering, also depend on surface texture. Accordingly, non-spherical shaped particles formed by the methods described herein with controlled surface texture also extremely useful in studies of shape-sensitive phenomena in physics, for example, light scattering, interfacial adsorption, packing densities, sedimentation, and fluidization and granular flows.

### EXAMPLES

The present invention may be further understood by reference to the following non-limiting examples.

### Example 1

### Particles Produced Using Scheme A

The fabrication conditions used to generate the particles discussed in this example are shown schematically in FIG. 1, Scheme A, and in Table 1.

TABLE 1
Fabrication conditions for particles reported in Example 1

| | Original | | Stretching | Film |
|---|---|---|---|---|
| | Sphere | | Aspect | Thickness |
| Particle | Diameter | Liquefaction | Ratio of | (μm), |
| Name | (μm) | Method | film | Plasticizer |
| (b) | 5.7 | 120° C. | 2 | 35, glycerol |
| rectangular | | | | |
| disks | | | | |
| (c) | 0.9 | 120° C. | 10.9 | 35, glycerol |
| rectangular | | | | |
| disks | | | | |
| (d) rods | 0.9 | 120° C. | 2.4 | 70, none |
| (e) rods | 0.9 | 120° C. | 5.5 | 70, none |
| (f) worms | 0.9 | 155° C. | 8.7 | 35, glycerol |
| (g) oblate | 2.9 | 125° C. | 2 (2D) | 35, glycerol |
| ellipses | | | | |
| (h) prolate | 0.9 | toluene | 1.1 | 35, glycerol |
| ellipses | | | | |
| (i) elliptical | 1.8 | toluene | 4.9 | 35, glycerol |
| disks | | | | |
| (j) UFOs | 5.7 | toluene | 2.3 (2D) | 35, glycerol |
| (k) circular | 2.9 | toluene | 1.9 (2D) | 35, glycerol |
| disks | | | | |

Results:

Simple stretching of particles in one dimension (1-D) led to the formation of several different shapes depending on the film properties and method of liquefaction. 1-D stretching of a 35 μm thick plasticized film at 120° C. produces rectangular disks. Increasing the amount of stretching increases the aspect ratio (length to width) but the shape is preserved. Film thickness had a profound impact on the shape of heat-stretched particles. Use of an unplasticized 70 μm thick film, under otherwise identical conditions, produces rods with a nearly circular cross-section. Extensive stretching makes the same shape with a larger aspect ratio. The aspect ratio of stretched particles can be continuously controlled from 1 to approximately 11 and is typically smaller than the extension ratio of the film itself. However, an exception was found when particles were stretched at high temperatures (155° C.) in a 35 μm thick plasticized film and worm-like particles were created. The exact shape of worms and their tortuosity varied from particle to particle. Two dimensional (2-D) stretching of heat-liquefied particles led to oblate ellipsoids with aspect ratios dictated by the extent of stretching.

Replacing heat by toluene as a mode of liquefying particles led to entirely different shapes. The exact reason is not clear, although the viscosity of polystyrene (“PS”), which is different in the two cases, is a possible reason. Moderate stretching of toluene-liquefied particles in 35 μm plasticized films resulted in prolate ellipsoids. However, increased stretching did not preserve the shape, as in heat stretching, and led to thin elliptical disks. Peculiar results were obtained when toluene-liquefied particles were stretched in 2-D. Moderate stretching of toluene-liquefied particles led to UFO-like particles. Extensive stretching under the same conditions or comparable stretching of smaller particles, however, eliminated the dome and produced flat circular disks. While the degree of stretching modulated only the aspect ratio of heat-liquefied particles, it actually changed the shape of toluene-liquefied particles.

### Example 2

### Particles Produced Using Scheme B

The fabrication conditions used to generate the particles discussed in this example are shown schematically in FIG. 1, Scheme B, and in Table 2.

TABLE 2
Fabrication conditions for particles reported in Example 2

| | Original | | Stretching | Film |
|---|---|---|---|---|
| | Sphere | | Aspect | Thickness |
| Particle | Diameter | Liquefaction | Ratio of | (μm), |
| Name | (μm) | Method | film | Plasticizer |
| (a) barrels | 2.9 | 130° C. | 1.6 | 35, none |
| (b) bullets | 2.9 | 140° C. | 1.6 | 35, none |
| (c) pills | 2.9 | toluene | 3, film | 35, glycerol |
| | | | dried off | |
| | | | stretcher | |
| (d) pulleys | 9 | toluene | 1.8 (2D) | 35, glycerol |
| (e) bi-convex | 0.9 | toluene | 1.8 (2D) | 35, glycerol |
| lenses | | | | |

Results

1-D stretching of the film without particle-liquefaction creates an ellipsoidal void around the particle. Upon heat-induced liquefaction, polystyrene fills the void in a temperature-dependent manner. At relatively low temperatures (130° C.), the particle remains in the middle of the void and results in a barrel-like structure upon solidification with concave regions at both ends. Interestingly, liquefaction at higher temperatures (140° C.), keeping all other parameters the same, favours distribution of polystyrene to one end of the void and forms bullet-like structures. Repeating the same procedure after 2-D stretching of the film produced oblate ellipsoids, much like those obtained using scheme A. Replacing heat by toluene as a means of liquefaction produced different shapes. 1-D stretching in air followed by toluene-induced liquefaction formed pill-like particles. 2-D stretching of the film in air followed by toluene-induced liquefaction led to pulley-shaped particles (a circular disk with a groove in the middle). Repeating the same procedure with extensive stretching produced bi-convex lenses.

SEMs demonstrated some of the shapes which may be generated using Scheme B from FIG. 1: barrels, bullets, pills, pulleys, and bi-convex lenses.

### Example 3

### Particles Produced Using Scheme C

The fabrication conditions used to generate the particles discussed in this example are shown in Table 3.

TABLE 3
Fabrication conditions for particles reported in Example 3

| | Original | | Stretching | Film |
|---|---|---|---|---|
| | Sphere | | Aspect | Thickness |
| Particle | Diameter | | Ratio of | (μm), |
| Name | (μm) | Procedure | film | Plasticizer |
| (a) ribbons | 2.9 | Stretch in air, liquify | 4, 4 | 35, |
| | | with toluene, dry in | | glycerol |
| | | air, reinforce with | | |
| | | PVA, stretch in air, | | |
| | | liquefy with toluene | | |
| (b) bicones | 0.9 | Start with elliptical | 3, 3 | 35, |
| | | disks, reinforce with | | glycerol |
| | | PVA, liquefy with | | |
| | | toluene, stretch | | |
| (c) | 2.9 | Start with elliptical | 3, 2 | 35, |
| diamond | | disks, reinforce with | | glycerol |
| disks | | PVA, liquefy with, | | |
| | | stretch along the | | |
| | | minor axis of | | |
| | | original elliptical | | |
| | | disks | | |
| (d) | 2.9 | Stretch in air, | 3, 2 | 35, |
| emarginate | | liquefy with toluene, | | glycerol |
| disks | | dry in air and | | |
| | | isopropanol, | | |
| | | reinforce with PVA, | | |
| | | stretch in air perp., | | |
| | | liquify with toluene | | |
| (e) flat pills | 2.9 | Stretch sequentially | 1.5, 1.5, | 35, |
| | | along both diagonals | 3, 4 | glycerol |
| | | in air, stretch along | | |
| | | the length, liquefy at | | |
| | | 120° C., cool to room | | |
| | | temperature, stretch | | |
| | | perp. at 120° C. | | |
| (f) | 0.9 | Stretch in air, | 3, 3 | 35, |
| elongated | | liquefy with toluene, | | glycerol |
| hexagonal | | dry in air and | | |
| disks | | isopropanol, stretch | | |
| | | perp in air, liquefy | | |
| | | with toluene | | |
| (g) ravioli | 2.9 | Start with barrels, | 1.4 | 35, none |
| | | stretch along the axis | | |
| | | perpendicular to the | | |
| | | length of the barrel, | | |
| | | liquefy with toluene | | |
| (h) tacos | 2.9 | start with barrels, | 1.4 | 35, none |
| | | stretch along the axis | | |
| | | perpendicular to the | | |
| | | length of the barrel | | |
| | | in air, liquefy at | | |
| | | 130° C. | | |

Results:

Scheme C of FIG. 1 involves the sequential use of various combinations of scheme A and scheme B. Combinations of schemes A and B led to even more unusual shapes. For example, 1-D stretching in scheme B with toluene followed by reinforcement of the film and repeated stretching according to scheme B led to ribbon-like particles with curled ends. Conversely, 1-D stretching of elliptical disks in a reinforced film produced according to scheme A produced bicones. Several additional shapes including diamond disks, emarginate disks, flat pills, elongated hexagonal disks, ravioli, and tacos were also made.

The method can be further modified to control additional design features such as surface texture while keeping size and shape constant. For example, in scheme B when the film was removed from the stretcher after stretching but prior to toluene liquefaction, wrinkled prolate ellipsoids and wrinkled oblate ellipsoids were formed after 1D and 2D stretching, respectively. In another example, porous elliptical disks were formed when toluene-liquefied particles, stretched according to scheme A, were immediately immersed in isopropyl alcohol to remove toluene, omitting the air drying step.

A cartoon summary of the shapes generated in Examples 1-3 is provided in FIG. 2.

### Example 4

### Effect of Shape on Internalization of Particles by Phagocytes

Materials and Methods

Continuous alveolar rat macrophage cells NR8383 (American Type Culture Collection (ATCC), Manassas, Va.) were used as model macrophages. Mouse peritoneal macrophage cells J774 were also used to verify the generality of results amongst macrophage populations of different species and tissues. Both cell types were cultured in F-12K media (ATCC) supplemented with 10% heat inactivated fetal bovine serum and 1% penicillin/streptomycin (Sigma Chemicals) under standard culture conditions (37° C., 5% CO₂, humidified). To ensure that macrophages were capable of spreading, cells were incubated on plain and IgG-coated coverslips and viewed with phase contrast light microscopy to identify circular spread cells.

Cells (2×10⁵ cells/mL) were allowed to attach in dishes lined with coverslip glass in F-12K media supplemented with 10% FBS and 25 mM HEPES (Sigma Chemicals). The dishes were placed on an Axiovert® 25 microscope (Carl Zeiss Inc., Thornwood, N.Y.) at 100× with phase contrast filters and equipped with Bioptechs Delta T Controlled Culture Dish System® (Bioptechs Inc., Butler, Pa.) to keep the cells at 37° C. Particles (1 particle per cell) were added to the dishes and bright-field images were collected every 30 seconds for 2 hours by a CoolSNAPHQ® CCD camera (Roper Scientific, Tucson, Ariz.) connected to the Metamorph® software. In some cases cells were observed for 12 hours and cell behavior was the same as for 2 hours. Observed cells were randomly chosen from the entire population thus discounting potential bias due to heterogeneity in macrophage size (radius 7.5±2.5 μm). Images were condensed into movies and analyzed manually for phagocytic events. Successful phagocytosis exhibited membrane ruffling at the site of attachment, blurring the crisp boundary of the membrane, and subsequent reforming of the membrane boundary after internalization. The method of visual scoring of phagocytosis was validated for IgG-opsonized particles using Alexa Fluor monkey anti-rabbit secondary fluorescent antibody that bound to rabbit IgG on particles when they were not internalized (Molecular Probes).

Results

Internalization of both opsonized and non-opsonized particles exhibited a strong dependence on local particle shape from the perspective of the phagocyte. Local shape varies not only for different particles but also for different points of initial contact on the same particle, except for spheres. For example, macrophages that attached to elliptical disks (major axis 14 μm, minor axis 3 μm) along the major axis (discussed quantitatively below) internalized them very quickly, in less than 6 minutes. The macrophage membrane was seen moving along the length of the particle in a coordinated, unified fashion. On the other hand, cells that attached to the same elliptical disks along the minor axis or flat side did not internalize them, even after 2 hours. They did, however, spread on the particle surface but with non-synchronized, separate fronts moving in different directions at different times. Macrophages attached to the flat side of IgG-opsonized elliptical disks exhibited more spreading than those attached to non-opsonized particles but the final result was the same, no phagocytosis. Since the particles used for these studies possessed identical properties (dimensions, surface area, volume, and chemistry), observations show that the local particle shape at the point of initial contact, not the overall size, determined their phagocytic fate. Similar results were seen for all shapes including UFO-shaped particles, where internalization does not occur when cells attach to the concave region but internalization does occur after attachment to the dome or ring region.

Time-lapse video micrographs spanning 39 minutes of macrophages interacting with identical non-opsonized elliptical disk particles (major axis 14 μm, minor axis 3 μm) from two different orientations a shows a macrophage that attaches to the major axis of an elliptical disk and shows a macrophage that attaches to the flat side of an elliptical disk.

SEMs were taken of macrophages interacting with particles and overlays of bright-field and fluorescent images of macrophages interacting with particles after fixing the cells and staining for polymerized actin with rhodamine phalloidin. An SEM was taken of a macrophage phagocytosing an elliptical disk which it interacted with initially along the minor axis of the elliptical disk. An SEM was taken of a macrophage spreading on an elliptical disk which it interacted with initially along the flat side of the elliptical disk. An SEM was taken of a macrophage phagocytosing a spherical particle. An overlay was prepared of bright-field and fluorescent images of a macrophage phagocytosing an elliptical disk which it interacted with initially along the minor axis of the elliptical disk, of bright-field and fluorescent images of a macrophage spreading on an elliptical disk which it interacted with initially along the flat side of the elliptical disk, and of bright-field and fluorescent images of a macrophage phagocytosing a spherical particle.

Scanning electron microscopy (SEM) images provided more evidence for an orientation bias for phagocytosis. Opsonized particles were incubated with alveolar macrophages. SEM was used for high magnification confirmation of cell membrane progression on the particles at various times during internalization. After 7 to 60 minutes of incubation with particles at 37° C., cells were fixed with 2% EM grade glutaraldehyde (Electron Microscopy Sciences, Hatfield, Pa.). They were washed with serial dilutions of water and ethanol, dried under vacuum, and coated with palladium (Hummer 6.2 Sputtering System). Cells were imaged with the Sirion 400 SEM at 2 eV.

The cell membrane showed marked progression on elliptical disks when approached along the major axis. In contrast, cells that attached to the flat side of elliptical disks exhibited spreading but no engulfment of particles, even after 2 hours. As a reference point, consistent engulfment was observed on spheres, whether opsonized or not.

Further insight for orientation-dependent particle phagocytosis was gained by staining macrophages for polymerized actin at various times during phagocytosis. Actin polymerization is the principal mechanism by which macrophages push the leading edge membrane and engulf particles (May and Machesky, J. Cell Sci., 114:1061-1077 (2001)). Initially an actin cup, comprised of a dense actin network, forms beneath the particle. As additional actin polymerization and remodeling occur and the membrane progresses, the actin cup is transformed into an actin ring around the particle that pushes the membrane along the particle until it is internalized (Lee, et al., Biochim. Biophys, Acta, 1525:217-227 (2001); Aizawa, et al., J. Cell Sci., 110:2333-2344 (1997); Cougoule, et al., Semin. Cell Dev. Biol., 15: 679-689 (2004)).

Cells were allowed to attach in dishes as in time-lapse video microscopy. Particles were added to the dishes and incubated at 37° C. for 10 or 120 minutes. The cells were fixed with 4% EM grade paraformaldehyde (Electron Microscopy Sciences) for 30 minutes. Once fixed, the cells were washed with PBS and permeabilized with 0.1% Triton X-100 (ICN Biomedicals, Inc., Aurora, Ohio) for 3 minutes. The cells were washed again with PBS and 2.5 units/ml rhodamine phalloidin (Molecular Probes) was added to each dish for 15 minutes to stain polymerized actin filaments. The dishes were washed with PBS and viewed at 100×. Bright-field and fluorescent images of cells with a single attached particle were acquired and overlayed. Cells were inspected manually for the presence of a fluorescent actin cup or ring.

Spheres and elliptical disks that attached to macrophages along the major axis exhibited an actin cup at short times that later transformed to a ring around the particle as phagocytosis progressed. Macrophage attachment to the flat side of elliptical disks, in spite of actin polymerization at points of contact and spreading, did not exhibit an actin cup or ring. Formation of an actin cup is a clear indicator of initialization of internalization and was observed only at certain local shapes.

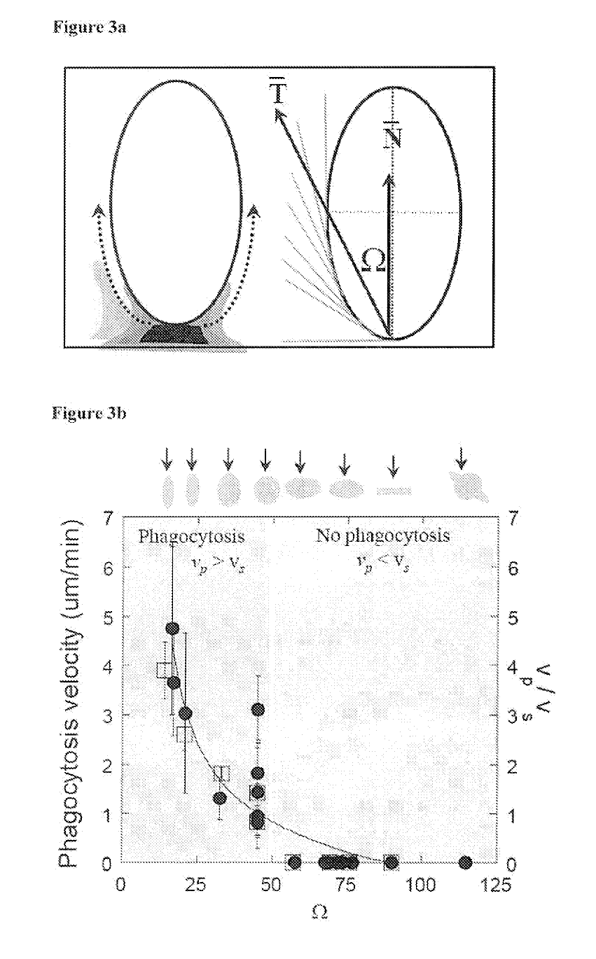

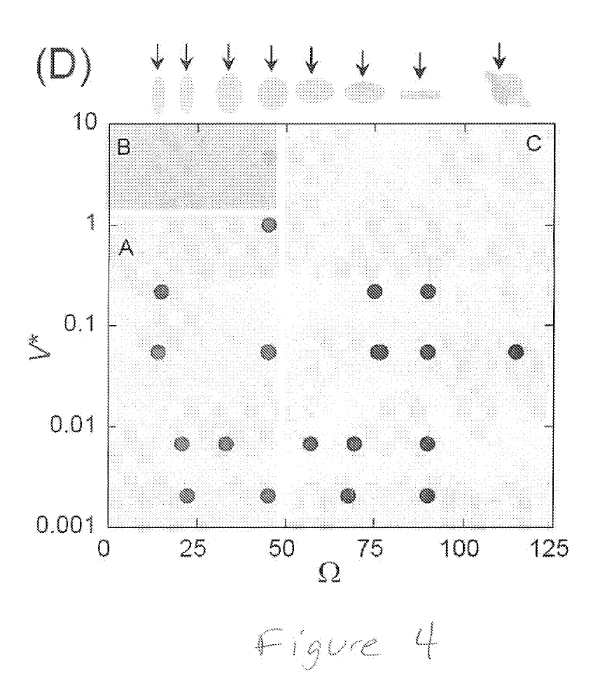

Referring to FIGS. 3 and 4, to arrive at a generalized and quantitative statement about the role of shape in phagocytosis, the angle Ω between the membrane normal was defined at the point of initial contact, N̅ and a vector T̅ , whose angle represents the mean direction of tangents drawn to the target contour from the point of initial contact to the center line of the target.

$$Ω = {cos}^{- 1} ( \overline{N} \cdot \overline{T} ) = {〈 {\int }_{0}^{θ} \frac{d s}{d θ} κ ( θ ) d θ 〉}_{0 , π / 2}$$

where κ(θ) is curvature and ds/dθ is the angular gradient of the arc length (http://mathworld.wolfram.com/Ellipse.html). θ=0 is defined as the point of contact. Ω, evaluated numerically for each case, is a dimensionless parameter and depends only on the particle's shape and its point of attachment to the macrophage. It indicates the mean angle made by the membrane with N̅ as it travels around the particle during phagocytosis. For each attachment site on a particle, there exist 2 values of Ω defined for two orthogonal views of the particle, the larger of which is used in further analysis. For all sized spheres, the dome or ring of UFOs, and the edge of OEs, Ω=45°. For an elliptical disk with a major axis a, minor axis b and relatively small thickness, Ω˜arctan(b/a) for a particle attaching along the major axis, Ω˜arctan(a/b) for attachment along the minor axis, and Ω≈90° for a cell attaching on the flat side. For the concave region of a UFO, Ω>90°. Since Ω depends only on particle shape, dependence of phagocytosis on size and shape can be clearly separated and understood.

Dependence of the rate of phagocytosis on Ω can be clearly seen in FIG. 3b where internalization velocity (total distance traveled by macrophage membrane to complete phagocytosis, evaluated in the two-dimensional projected view of the particle, divided by the time required to complete phagocytosis) is plotted against Ω. Phagocytosis velocity decreased with increasing Ω. Furthermore, there was an abrupt transition in internalization velocity to zero at Ω˜45°. Zero velocity is assigned when phagocytosis is not completed within the period of observation (2 hours). Any lack of internalization in FIG. 3b is not due to particle size since all particles in this figure were successfully internalized from at least one attachment orientation. IgG-coated particles exhibited the same Ω-dependence as non-opsonized particles, confirming the generality of the dependence of phagocytosis on particle shape.

The sudden transition from phagocytosis to only spreading at Ω˜45° is rather striking. Particles with Ω>45° induced significant spreading of cells but not internalization. Therefore, the fine line between phagocytosis and spreading is defined by the shape of the particle from the cell's perspective, Ω.

The overall process of phagocytosis is a result of the complex interplay between shape and size. The phase diagram in FIG. 4 shows whether or not internalization was initialized and completed for particles with different combinations of Ω and V*, the ratio of particle volume to macrophage volume. Initiation of internalization was judged by formation of an actin cup or ring and completion was judged by closure of the membrane. The diagram shows three regions: the successful phagocytosis region (Ω≦45°, V*≦1) where phagocytosis is initiated and completed quickly, the attempted phagocytosis region (Ω≦45°, V*>1) where phagocytosis is initiated but not completed within the period of observation, and the spreading region (Ω>45°) where particle attachment takes place and macrophages spread on the particle but phagocytosis is not initiated. This diagram shows that initiation of phagocytosis is governed by Ω while V* primarily influences completion. Macrophages phagocytosed particles as large as themselves when approached from the preferred orientation (Ω≦45°). However, when approached from the undesired orientation (Ω>45°), they did not internalize particles with volumes as small as 0.2% of the cell volume.

### Example 5

### Phagocytosis of Non-Spherical Particles

Particles were fabricated with specific shapes (barrels and worms) and their phagocytosis was studied. Macrophages were not able to phagocytose these particles whereas they readily ingested spheres of the same volume. With barrels, the shape is such that for most points where the macrophage attaches to the particle, the value of Ω is greater than 45°. This significantly reduced phagocytosis as expected, since previous experiments predict decreased phagocytosis for Ω greater than 45°. For worms, Ω is greater than 45° for most points except for the very tip. However, given the small area of the tip, the likelihood of macrophage attaching to it is very low. Thus, worm-like particles are very difficult to phagocytose. Drugs can be encapsulated in barrel and worm-shapes particles and delivered for various applications.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of making non-spherical particles comprising
providing polymeric nano or micro particles in a polymeric film and applying physical manipulation of the polymeric film to change the shape of the particles.

2. The method of claim 1, further comprising
liquefying the nano or micro particles in the polymeric film and resolidifying the particles after manipulation of the film.

3. The method of claim 2 wherein the particles are liquefied by adding a solvent in which the polymeric film is immiscible or by applying heat.

4. The method of claim 1 wherein the film is manipulated in a first direction.

5. The method of claim 4 wherein the film is manipulated in a second direction.

6. The method of claim 1 wherein the particles adhere to the polymer film via ionic bonds or hydrogen binding.

7. The method of claim 1 wherein the polymeric film comprises a plasticizer.

8. A polymeric film comprising non-spherical nano or micro particles, wherein the polymeric film comprises a different polymer than the nano or micro particles.

9. The film of claim 8, wherein the film is prepared by a method comprising
providing spherical nano or micro particles in a polymeric film and applying physical manipulation of the polymeric film to change the shape of the particles.

10. Non-spherical nano or micro particles prepared by a method comprising
providing spherical nano or micro particles in a polymeric film and applying physical manipulation of the polymeric film to change the shape of the particles.

11. The particles of claim 10 further comprising a prophylactic, therapeutic or diagnostic agent.

12. The particles of claim 10 further comprising a targeting agent.

13. A method of treating an organism by administering at least one therapeutic agent encapsulated in at least one non-spherical nano or micro particle.

14. The method of claim 13 where the therapeutic agent and particle material are the same.

15. The method of claim 13 where the particles are administered orally, intravenously, nasally, pulmonarily, rectally or topically.

16. A method of diagnosing an organism by administering at least one diagnostic agent encapsulated in at least one non-spherical nano or microparticle.
